(19) United States
(12) Patent Application Publication
(10) Pub. No.: US 2025/0082304 A1
(43) Pub. Date: Mar. 13, 2025

(54) METHOD AND SYSTEM FOR GUIDING A USER HOLDING IN ONE HAND AN ULTRASOUND OBSERVATION PROBE TOWARDS A PRE-RECORDED ACOUSTIC WINDOW

(71) Applicants: Philippe ARBEILLE, Lignieres de Touraine (FR); CENTRE NATIONAL D'ÉTUDES SPATIALES, Paris (FR); MEDES-INST MEDECINE PHYSIOLOGIE SPATIALE, Toulouse (FR)

(72) Inventors: Philippe ARBEILLE, Lignieres de Touraine (FR); Didier CHAPUT, Labarthe sur Leze (FR); François DERACHE, Toulouse (FR); Orphée FAUCOZ, Toulouse (FR); Aristée THEVENON, Toulouse (FR)

(73) Assignees: Philippe ARBEILLE, Lignieres de Touraine (FR); CENTRE NATIONAL D'ÉTUDES SPATIALES, Paris (FR); MEDES-INST MEDECINE PHYSIOLOGIE SPATIALE, Toulouse (FR)

(21) Appl. No.: 18/722,399

(22) PCT Filed: Dec. 14, 2022
(86) PCT No.: PCT/FR2022/052359

§ 371 Date: Jun. 20, 2024

(30) Foreign Application Priority Data
Dec. 20, 2021 (FR) FR2114051

(51) Int. Cl. A61B 8/00

(52) U.S. Cl. CPC A61B 8/4254

(57) ABSTRACT

A method for guiding a user holding in one hand a probe for ultrasound observation a patient, including: placing a support on the body surface; acquiring at least one image; determining the reference frame; determining coordinates of at least one vertex of a polygon placed in a first plane transverse to the recorded direction of the probe; displaying the at least one image; projecting on the at least one displayed image; determining the current position and the current direction of the probe in the reference frame; projecting, on the at least one displayed image, the at least one vertex; bringing the probe to a new current position and a new current direction, the new current position and the new current direction coinciding with the recorded position and the recorded direction.

The present invention concerns the field of the use of a probe configured for ultrasound observation of a patient.

It is important, to make a correct diagnosis, that the observation probe is correctly positioned. However, if an ultrasound expert knows how to manipulate the probe to find the appropriate acoustic window, i.e. the position and the direction of the probe which will allow the considered organ to be observed under satisfactory conditions, the same is not true for a novice user who will find it difficult to find the correct direction and position of the probe in order to obtain a usable image for a medical diagnosis.

The novice user needs help finding the good position and the good orientation of the probe that an expert user has previously saved.

The object of the invention is therefore to propose a solution to all or part of these problems.

To this end, the present invention concerns a method for guiding a user holding in one hand a probe configured for ultrasound observation of a patient's body, the probe being provided with a first object secured to the probe, the first object comprising first markers on the surface of the first object, the probe being held in a current position and along a current direction, a display device configured to receive at least one image acquired by a camera configured to observe in its field the probe, the first object, and a second object comprising second markers on the surface of the second object, the user being guided to a recorded position and a recorded direction of the probe, the method comprising the following steps:
placing a support at a predetermined point of the surface of the patient's body observed with the probe, the support being configured to receive on a surface of the support the second object while maintaining the second object in a predetermined orientation and position of the second object relative to a fixed reference frame relative to the patient's body;
placing the second object on the surface of the support, so that the second object is maintained in a predetermined orientation and position relative to the fixed reference frame relative to the patient's body.
acquiring at least one image with the camera;
determining the reference frame, from the second markers identifiable by processing the at least one image, the recorded position and the recorded direction of the probe being defined in said reference frame;
in the reference frame, determining coordinates of at least one first point corresponding to a vertex of a polygon disposed in a first plane transverse to the recorded direction of the probe, so that a central area of the polygon is crossed by a straight line parallel to the recorded direction of the probe and passing through the recorded position of the probe;
displaying the at least one image on the display device;
projecting, in augmented reality on the at least one displayed image, the at least one first point;
determining the current position and the current direction of the probe in the reference frame, from the first markers identifiable by another processing of the at least one image;
determining coordinates in the reference frame of at least one second point, the at least one second point corresponding to the vertex of the polygon disposed in a second plane transverse to the current direction of the probe, so that a central area of the polygon is crossed by a straight line parallel to the current direction of the probe and passing through the current position of the probe;
projecting, in augmented reality on the at least one displayed image, the at least one second point;
bringing the probe to a new current position and a new current direction so that a new projection in augmented reality of the at least one second point corresponding to the vertex of the polygon disposed in a new second plane transverse to the new current direction of the probe, a central area of the polygon being crossed by a straight line parallel to the new current direction of the probe and passing through the new current position of the probe, the new projection being closer to the projection of the at least one first point, than the projection of the at least one second point, until the at least one second point and the at least one first point coincide, and so that the new current position and the new current direction coincide with the recorded position and the recorded direction, the steps of projection and new projection in augmented reality of the at least one second point being carried out in real-time as the probe moves.

According to these provisions, the second user is guided by the display of the projections in augmented reality to gradually cause the new current position and the new current direction to coincide with the recorded position and the recorded direction, the recorded position and the recorded direction corresponding to ideal position and orientation of the probe to obtain a good view of a specific organ.

According to one implementation, the invention comprises one or more of the following characteristics, alone or in a technically acceptable combination.

According to one implementation, the first object is a first cube comprising the first markers on the faces of the cube.

According to one implementation, the first cube is a first QR cube.

According to one implementation, the second object is a second cube comprising the second markers on the faces of the second cube.

According to one implementation, the second cube is a second QR cube.

According to one implementation:
the at least one first point comprises four first points each corresponding to four vertices of the polygon disposed in the first plane transverse to the recorded direction of the probe, so that the central area of the polygon is crossed by the straight line parallel to the recorded direction of the probe and passing through the recorded position of the probe,
and
the at least one second point comprises four second points each corresponding to the four vertices of the polygon disposed in the second plane transverse to the current direction of the probe, so that the central area of the polygon is crossed by the straight line parallel to the current direction of the probe and passing through the current position of the probe.

According to one implementation, the polygon is a square.

According to one implementation:
a first geometric shape is associated with the at least one first point, the first geometric shape being projected in augmented reality during the projection step, in augmented reality on the at least one displayed image, of the at least one first point, and
a second geometric shape is associated with the at least one second point, the second geometric shape being projected in augmented reality during the projection step, in augmented reality on the at least one displayed image, of the at least one second point.

According to these provisions, the second user is guided more effectively by the display of the projections in augmented reality of the first and second more visible geometric shapes.

According to one implementation, the first geometric shape is a cube, respectively a sphere, centered around the at least one first point, the second geometric shape is a sphere, respectively a cube, centered around the at least one second point.

According to these provisions, it is possible to overcome the occlusion problems often encountered in the applications in augmented reality.

According to one implementation, a straight-line segment connecting the at least one first point to the at least one second point is displayed as a function of a distance between the at least one first point and the at least one second point.

According to one implementation, the straight-line segment is displayed in a color representative of the distance measured in the reference frame.

According to one implementation, the predetermined point of the surface of the body is predetermined so that a position and an orientation, in the fixed reference frame relative to the patient's body, of the surface of the support disposed at the predetermined point is stable over time, and repeatable, i.e. when the step of placing the support at the predetermined point of the surface of the patient's body is repeated several times in succession, a standard deviation of the position of the surface of the support is less than one deviation in position and a standard deviation of the orientation of the surface of the support is less than a deviation in orientation.

According to one implementation, the deviation in position is equal to 10 mm, preferably equal to 5 mm, preferably equal to 0 mm.

According to one implementation, the deviation in orientation is equal to 2°, preferably equal to 1°, preferably equal to 0°.

According to one implementation, the predetermined point of the surface of the body is a point on the sternum, located close to the Procelus Xifoidan, i.e. a lower end of the sternum. Thus, disposing the support in this particular place makes it possible to obtain a particularly stable and repeatable orientation and position of the surface of the support and, consequently, of the second object maintained on the surface of the support.

In one implementation, the support has an elongated shape along a direction of elongation of the support, so that the support placed at the predetermined point is aligned between the sternum and the clavicle. In this way, the support conforms the body of the majority of patients, and reinforces the stability of the reference object placed on the support, particularly in the microgravity conditions associated with one of the intended uses of the device in space.

According to one implementation, in a direction transverse to the direction of elongation of the support, the support has a dimension comprised between 3.5 cm and 10 cm, preferably comprised between 3.5 cm and 7 cm, preferably comprised between 3.5 cm and 5 cm. Thus, when the support is aligned between the sternum and the clavicle, it is possible for the sonographer, without compromising stability, to reach all the acoustic areas of the organs, whereas a slightly wider shape would handicap the ultrasound of the heart area in parasternal view. In particular, the dimension in the direction transverse to the direction of elongation is minimum at the patient's heart to allow ultrasound of the area of the heart in parasternal view, while it is greater at the predetermined point at the sternum on which the support is disposed, to promote stability.

According to one implementation, the support is flexible so as to match the shape of the thorax by coming to be disposed on the sternum from top to bottom, with at its base a surface configured to receive and maintain the second object in stable and repeatable orientation and position, defined by the stable and repeatable orientation and position of the surface of the support. When the second object is a QR cube, the surface of the support has a square shape corresponding to one of the faces of the QR cube.

According to one implementation, a distance between the predetermined point and the lower end of the sternum is less than 10 mm, preferably less than 5 mm, preferably less than 2 mm.

According to one implementation, the recorded position and the recorded direction of the probe being defined by a first user when the probe is held in the hand of the first user in a current position and along a current direction, the step of determining the current position and the current direction of the probe in the reference frame, from the second identifiable markers on the at least one image comprising a step of recording the current position of the probe and the current orientation.

The invention also concerns a system for guiding a user holding in one hand a probe configured for ultrasound observation of a patient's body, the system comprising the probe provided with a first object secured to the probe, the first object comprising first markers on the surface of the first object, the system also comprising a second object comprising second markers on the surface of the second object, the system comprising a support placed at a predetermined point of the surface of the patient's body observed with the probe, the second object being placed on a surface of the support configured to maintain the second object in predetermined orientation and position of the second object relative to a fixed reference frame relative to the patient's body, the system further comprising a camera configured to observe in its field the probe, the first object, and the second object, the system comprising a display device configured to receive at least one image acquired by the camera, the display device being configured to display the at least one image acquired by the camera, and to display in the at least one image in augmented reality at least one first point corresponding to a vertex of a polygon disposed in a first plane transverse to a recorded direction of the probe, so that a central area of the polygon is crossed by a straight line parallel to the recorded direction of the probe and passing through the recorded position of the probe, and to display in the at least one image in augmented reality at least one second point corresponding to the vertex of the polygon disposed in a second plane transverse to the current direction of the probe, so that a central area of the polygon is crossed by a straight line parallel to the current direction of the probe and passing through the current position of the probe, the user holding the probe in one hand being guided by the display to coincide the at least one first point with the at least one second point in the at least one displayed image, moving the probe to a new current position and a new current direction so as to gradually make the new current position and the new current direction of the probe coincide with the recorded position and the recorded direction of the probe.

According to one embodiment, the first object is a QR cube, the second object is a QR cube, the polygon is a square, the at least one first point comprises four points, and the at least one second point comprises four points.

For its good understanding, one embodiment and/or implementation of the invention is described with reference to the attached drawings representing, by way of non-limiting example, one embodiment or implementation respectively of a device and/or a method according to the invention. The same references in the drawings designate similar elements or elements whose functions are similar.

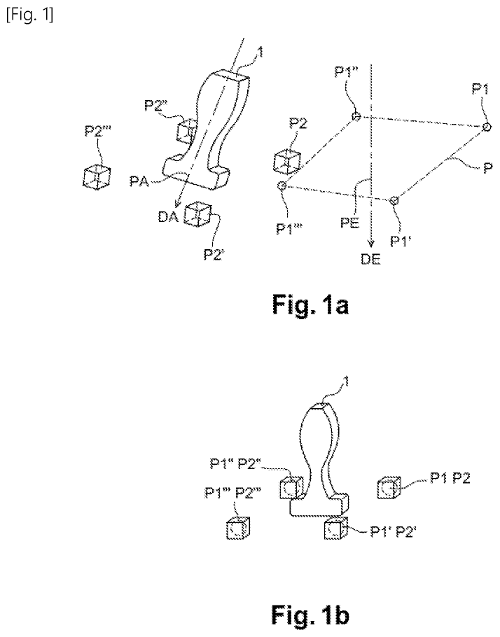

FIG. 1 is a perspective view of a probe in current direction and position, the current direction and position of the probe being displayed in real-time on a device for displaying an image of the probe, with four spheres, embedded in the image, at the vertices of a square located in a plane transverse to the current direction of the probe and four cubes at the vertices of the square in a plane transverse to a recorded direction corresponding to ideal position and orientation of the probe (cf. FIG. 1a); in FIG. 1b, the user, guided by the display of the four cubes according to FIG. 1a, rejoined the recorded orientation and position by moving the probe and the four spheres so as to bring the four spheres inside the four cubes in the displayed image.

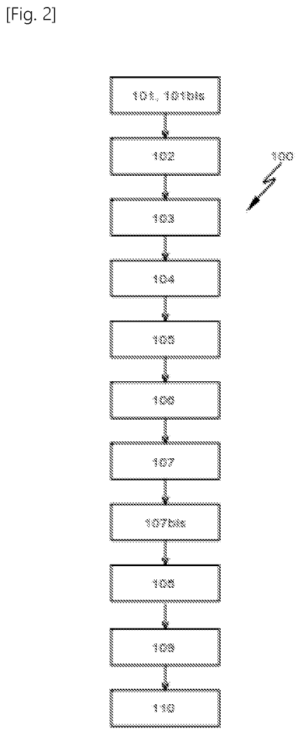

FIG. 2 is a schematic representation of the sequencing of the steps of the method according to the invention.

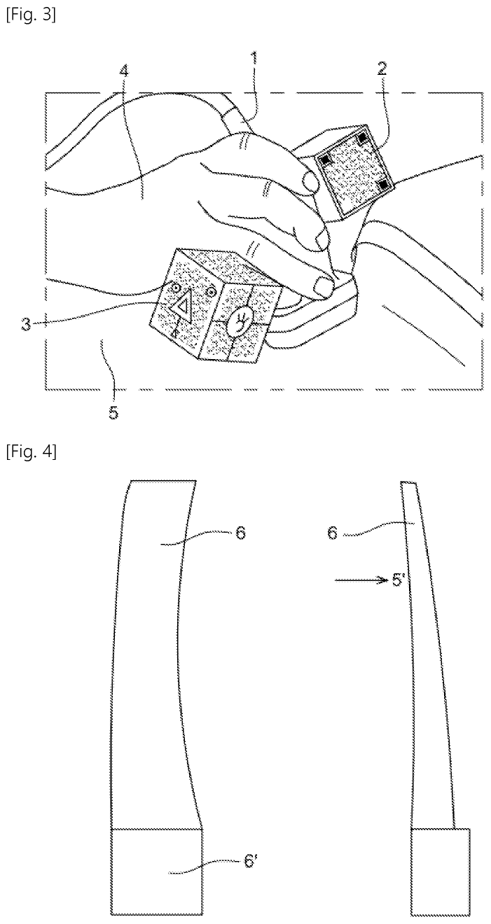

FIG. 3 is an image of the probe held in the hand of the user, the probe being provided with a first QR cube, secured to the probe, the first QR cube comprising first markers on its surface, a second QR cube comprising second markers on its surface, being configured to be placed on the surface of a support, not represented in FIG. 3, placed on a point of the sternum which allows a good calibration.

FIG. 4 is a top view (cf. FIG. 4a) and a side view (cf. FIG. 4b) of a support according to the invention.

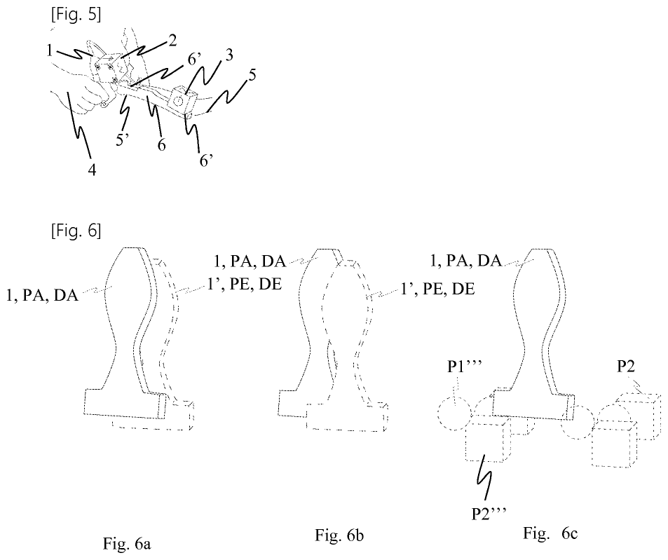

FIG. 5 is a perspective view of an elongated support according to one implementation of the invention, configured to be placed aligned between the sternum and the clavicle, at a predetermined point of the patient's body.

FIG. 6 is an illustration of the occlusion phenomenon: in FIG. 6a are presented on the one hand, in solid lines, the probe in its current position, and on the other hand, in broken lines, the probe in its recorded target position; in FIG. 6b the probe in its recorded target position, projected in augmented reality on the display device, partially obscures the target in its current position; FIG. 6c presents the display mode according to the invention which makes it possible to avoid obscuration.

The object of the method 100 is to guide a novice user to reach a good position and a good orientation of a probe 1 for the observation by ultrasound of a patient's organ. The good position and the good orientation of the probe has previously been determined by an experienced user, i.e. an expert, and has been saved, for example during a recording step 107bis, in the form of coordinates of a position PE of the probe and angular coordinates of a direction DE of the probe in a reference frame.

For this, as illustrated in FIG. 3, the probe 1 held in the hand of the user is provided with a first object 2 secured to the probe 1, the first object 2 comprising first markers on the surface of the first object 2. For example, the first object 2 is a QR cube, i.e. a cube comprising frames represented on the different faces of the cube

A camera is configured to observe in its field of observation the probe 1, the first object 2 comprising first markers, for example a first QR cube 2, and a second object 3 comprising second markers, for example a second QR cube 3. The second QR cube 3 is maintained in predetermined orientation and position relative to a reference frame by being placed 101bis on a support which has been placed 101 at a predetermined point 5′ of the surface 5 of the patient's body observed with the probe, the reference frame being fixed relative to the patient's body.

The camera is configured to acquire 102 one or more images of the probe 1, of the first object 2 comprising first markers, for example of the first QR cube 2, and of the second object 3 comprising second markers, for example of the second QR cube 3. A display device is associated with the camera to display 105 the image(s) acquired by the camera.

From the second markers of the second object 3, for example the second QR cube 3, observed and identified by processing the image(s) acquired by the camera, the reference frame is determined 103. The recorded position PE and the recorded direction DE of the probe 1 for good ultrasound observation of the patient's organ, recorded for example during the recording step 107bis, are defined in the reference frame; the recorded position PE and the recorded direction DE of the probe 1 may thus be projected in augmented reality, i.e. as an overlay on the acquired image(s).

For this, as illustrated in FIG. 1, in a first plane transverse to the recorded direction DE of the probe 1, a polygon P, for example a square, is determined by its vertices P1, P1′, P1″, P1′″; the coordinates of the vertices P1, P1′, P1″, P1′″ are determined 104 in the reference frame, so that the vertices P1, P1′, P1″, P1′″ are projected 106, in augmented reality on the displayed image(s).

The polygon P, in the implementation example presented here, is a square, but those skilled in the art will understand that the polygon may take other shapes, regular or irregular, in particular with more than four vertices, for example hexagon, octagon, etc.

In particular, in the step of projecting 106 in augmented reality the vertices P1, P1′, P1″, P1′″ of the polygon P, a geometric shape may be associated with each of the vertices P1, P1′, P1″, P1′″ of the polygon P and projected 106 in augmented reality on the image(s) acquired and displayed on a display device available to the user. Said geometric shape is for example a cube or a sphere, centered around each of the vertices P1, P1′, P1″, P1′″ of the polygon P. According to these provisions, the recorded position and the recorded direction of the probe 1 are displayed more visibly on the image(s) of the probe 1 presented to the user on the display device.

From the first markers present on the first object 2 secured to the probe 1, and identifiable by another processing of the image(s), the current position PA and the current direction DA of the probe 1 held in the hand 4 of the user is determined 107 in the reference frame. In a second plane transverse to the current direction DA of the probe 1, the polygon P, a square according to the example illustrated, is determined by its vertices P2, P2′, P2″, P2′″; the coordinates of the vertices P2, P2′, P2″, P2′″ of the polygon P in said second transverse plane, are determined 108 in the reference frame, so that the vertices P2, P2′, P2″, P2′″ are then projected 109, in augmented reality, on the displayed image(s).

As previously for the projection 106 of the vertices P1, P1′, P1″, P1′″ of the polygon P representative of the recorded position PE and the recorded direction DE of the probe 1, a geometric shape may be associated with each of the vertices P2, P2′, P2″, P2′″ of the polygon P in said second transverse plane, representative of the current position PA and of the current direction DA of the probe 1, in order to be projected 109 in augmented reality on the image(s) acquired and displayed on a display device available to the user. Said geometric shape is for example a sphere or a cube, centered around each of the vertices P2, P2′, P2″, P2′″ of the polygon P in said second transverse plane. According to these provisions, the current position PA and the current direction DA of the probe 1 is displayed in a more visible manner on the image(s) of the probe 1 presented to the user on the display device, at the same time as the recorded position PE and the recorded direction DE of the probe 1.

The association and the projection 106, 109 in augmented reality on the image(s) acquired and displayed on the display device available to the user, of a first geometric shape, at each of the vertices P1, P1′, P1″, P1′″ representative of the recorded target position of the probe, and respectively of a second geometric shape, different from the first geometric shape, at each of the vertices P2, P2′, P2″, P2′″ of the polygon P representative of the current position of the probe, has the advantage of making it possible to overcome the occlusion problems usually encountered in the augmented reality applications. Indeed, if the entire virtual probe 1′, in the position PE and the recorded target direction DE, shown in FIG. 6a of FIG. 6, was projected in augmented reality on the display device, it would at least partly obscure the real probe 1, in its current position PA, and its current direction DA, as shown in FIG. 6b of FIG. 6. The use of simplified geometric shapes to represent the current and recorded positions and directions of the probe, makes it possible to avoid this occultation, as is illustrated in FIG. 6c of FIG. 6.

According to an implementation of the invention, the first geometric shape associated with each of the vertices P1, P1′, P1″, P1′″ representative of the recorded target position of the probe is a cube, and the second geometric shape associated with each of the vertices P2, P2′, P2″, P2′″ of the polygon P representing the current position of the probe is a sphere. FIG. 1a illustrates another implementation in which the first geometric shape associated with each of the vertices P1, P1′, P1″, P1′″ representing the recorded target position of the probe is a sphere, and the second associated geometric shape at each of the vertices P2, P2′, P2″, P2′″ of the polygon P representing the current position of the probe is a cube.

In particular, for example, a line segment connecting each vertex P1, P1′, P1″, P1′″ of the polygon P in the first transverse plane, to the corresponding vertex P2, P2′, P2″, P2′″ of the polygon P in the second transverse plane is displayed so as to represent, for example according to a color code, a distance between each vertex P1, P1′, P1″, P1′″ and the corresponding vertex P2, P2′, P2″, P2′″. According to these provisions, the user can deduce more easily in what way he can displace the probe 1 to bring it 110 to a new current position and a new current direction so that a new projection in augmented reality of the vertices P2, P2′, P2″, P2′″ of the polygon P disposed in a new second plane transverse to the new current direction of the probe is closer to the current projection of the vertices P2, P2′, P2″, P2′″, than the projection of the corresponding vertices P2, P2′, P2″, P2′″ of the polygon P in the second transverse plane, until the corresponding vertices P2, P2′, P2″, P2′″ of the polygon P in the second transverse plane and the vertices P1, P1′, P1″, P1′″ of the polygon P in the first transverse plane coincides, and thus the new current position and the new current direction coincide with the recorded position PE and the recorded direction DE. The steps of projection 109 and new projection in augmented reality of the at least one second point P2, P2′, P2″, P2′″ being carried out in real time as the probe 1 is displaced.

According to an implementation mode of the method 100, the predetermined point 5′ for placing 101 the support 6, on the surface 5 of the body, is defined so that, in a fixed reference frame relative to the patient body, a position and orientation of the surface of the support, disposed at said predetermined point, is stable over time, and repeatable, i.e. when the step of placing 101 the support 6 at the predetermined point 5′ of the surface 5 of the patient body is repeated several times in a row, a standard deviation of the position of the surface 6′ of the support 6 is less than a deviation in position and a standard deviation of the orientation of the surface 6′ of the support 6 is less than a deviation in orientation.

Preferably, the deviation in position is equal to 10 mm, for example equal to 5 mm, in particular equal to 0 mm.

Preferably, the deviation in orientation is equal to 2°, for example equal to 1°, in particular equal to 0°.

More particularly, the predetermined point 5′ of the surface of the body is a point of the sternum, located near the Procelus Xifoidan, i.e. of a lower end of the sternum. Thus, disposing the support 6 in this particular place makes it possible to obtain a particularly stable and repeatable position and orientation of the surface 6′ of the support and, consequently, of the second object 3 maintained on the surface 6′ of said support. Indeed, at this place of the human body, it is possible to give an elongated shape to the support which allows alignment of the support between the sternum and the clavicle, thus conforming to the body of the majority of patients, and thus reinforcing the stability of the reference object placed on the support, in particular in the microgravity conditions associated with one of the envisaged uses of the device in space. Furthermore, at this place in the human body, it is possible, without compromising stability, to give the support a sufficiently narrow shape so that the sonographer can reach all the acoustic areas of the organs, whereas a slightly wider shape would handicap the ultrasound of the heart area in parasternal view. An example of the support 6 with an elongated shape placed in alignment with the support 6 between the sternum and the clavicle is presented in FIG. 5.

For example, in a direction transverse to a direction of elongation of the support, the support has a dimension comprised between 3.5 cm and 10 cm, preferably comprised between 3.5 cm and 7 cm, preferably comprised between 3.5 cm and 5 cm. Thus, when the support is aligned between the sternum and the clavicle, it is possible for the sonographer, without compromising stability, to reach all the acoustic areas of the organs, whereas a slightly wider shape would handicap the ultrasound of the heart area in parasternal view. In particular, the dimension in the direction transverse to the direction of elongation is minimum at the level of the patient heart to allow ultrasound of the heart area in parasternal view, while it is greater at the predetermined point at the level of the sternum on which the support is disposed, to promote stability.

In particular, as illustrated in FIG. 4, the support 6 is flexible so as to conform to the shape of the thorax by being placed on the sternum from top to bottom, with at its base a surface 6′ configured to receive and maintain the second object 3 in a particularly stable and repeatable position and orientation, defined by the particularly stable and repeatable position and orientation of the surface 6′ of the support 6. The surface 6′ configured to receive and maintain the second object can be at the bottom or at the top of the support 6, the bottom or the top being defined in relation to the body of the patient, whose head is deemed to be at the top and the feet, at the bottom. For example in FIG. 5, the reference 5 in this figure designates the patient's Adam's apple, and the second object 3 rests on the surface 6′ of the support 6, which is here at the top of the support 6; another surface 6′ is also arranged at the bottom of the support 6, to receive the second object 3. More particularly, when the second object 3 is a QR cube 3, the surface 6′ has a square shape corresponding to one of the faces of the QR cube 3.

According to one embodiment, a distance between the predetermined point 6′ and the lower end of the sternum is less than 10 mm, preferably less than 5 mm, preferably less than 2 mm.

According to one aspect, the invention concerns a system for guiding a user holding in one hand 4 a probe 1 configured for ultrasound observation of a patient's body, the system comprising the probe 1 provided with a first object 2 secured to the probe 1, the first object 2 comprising first markers on the surface of the first object 2. Said system also comprises a second object 3 comprising second markers on the surface of the second object. Said system comprises a support 6 placed at a predetermined point 5′ of the surface 5 of the patient's body observed with the probe 1, the second object 3 being placed on a surface 6′ of the support 6 configured to maintain the second object 3 in a position and a predetermined orientation of the second object 3 relative to a fixed reference frame relative to the patient's body. Said system further comprises a camera configured to observe in its field the probe 1, the first object 2, and the second object 3. Said system comprises a display device configured to receive at least one image acquired by the camera, the display device being configured to display the at least one image acquired by the camera, and to display in the at least one image, in augmented reality, at least one first point P1, P1′, P1″, P1′″ corresponding to a vertex of a polygon P placed in a first plane transverse to a recorded direction DE of the probe, so that a central area of the polygon is crossed by a straight line parallel to the recorded direction of the probe and passing through the recorded position of the probe 1. The display device is further configured to display in the at least one image, in augmented reality, at least one second point P2, P2′, P2″, P2′″ corresponding to the vertex of the polygon P disposed in a second plane transverse to the current direction of the probe, so that a central area of the polygon is crossed by a straight line parallel to the current direction of the probe and passing through the current position of the probe. So that the user holding the probe in one hand 4 is guided by the display to make the at least one first point P1, P1′, P1″, P1′″ coincide with the at least one second point P2, P2′, P2″, P2′″ in the at least one displayed image, by displacing the probe to a new current position and a new current direction by progressively making the new current position and the new current direction of the probe 1 coincide with the recorded position PE and the recorded direction DE of the probe 1.

For example, the first object 2 of said system is a first QR cube, the second object 3 of said system is a second QR cube, the polygon P is a square, the at least one first point comprises four vertices of the square, and the at least one second point comprises four vertices of the square.

1. A method for guiding a user holding in one hand a probe configured for ultrasound observation of a body of a patient, the probe being provided with a first object secured to the probe, the first object comprising first markers on the surface of the first object, the probe being held in a current position and according to a current direction, a display device configured to receive at least one image acquired by a camera configured to observe in its field the probe, the first object, and a second object comprising second markers on the surface of the second object, the user being guided towards a recorded position and a recorded direction of the probe, the method comprising the following steps:
placing a support at a predetermined point of a surface of the patient's body observed with the probe, the support being configured to receive on a surface of the support the second object by maintaining the second object in a predetermined position and orientation of the second object relative to a fixed reference frame relative to the patient's body;
placing the second object on the surface of the support, so that the second object is maintained in a predetermined position and orientation relative to the reference frame fixed relative to the patient's body;
acquiring at least one image with the camera;
determining the reference frame, from the second markers identifiable by processing the at least one image, the recorded position and the recorded direction of the probe being defined in the reference frame;
in the reference frame, determining coordinates of at least one first point corresponding to a vertex of a polygon disposed in a first plane transverse to the recorded direction of the probe, such that a central area of the polygon is crossed by a straight line parallel to the recorded direction of the probe and passing through the recorded position of the probe;
displaying the at least one image on the display device;
projection, in augmented reality on the at least one displayed image, the at least one first point;
determining the current position and the current direction of the probe in the reference frame, from the first markers identifiable by another processing of the at least one image;
determining coordinates in the reference frame of at least one second point, the at least one second point corresponding to the vertex of the polygon placed in a second plane transverse to the current direction of the probe, so that a central area of the polygon is crossed by a straight line parallel to the current direction of the probe and passing through the current position of the probe:
projecting, in augmented reality onto the at least one displayed image, the at least one second point;
bringing the probe into a new current position and a new current direction so that a new projection in augmented reality of the at least one second point corresponding to the vertex of the polygon disposed in a new second plane transverse to the new current direction of the probe, a central area of the polygon being crossed by a straight line parallel to the new current direction of the probe and passing through the new current position of the probe, the new projection being closer to the projection of the at least one first point than to the projection of the at least one second point, until the at least one second point and the at least one first point coincide, and thus the new current position and the new current direction coincide with the recorded position and the recorded direction, the projection and new projection in augmented reality steps of the at least one second point being produced in real time as the probe is displaced.

2. The method according to claim 1, wherein the first object is a first cube comprising the first markers on the faces of the cube.

3. The method according to claim 1,
wherein the second object is a second cube comprising the second markers on the faces of the second cube.

4. The method according to claim 1, wherein:
the at least one first point comprises four first points each corresponding to four vertices of the polygon disposed in the first plane transverse to the recorded direction of the probe, so that the central area of the polygon is crossed by the straight line parallel to the recorded direction of the probe and passing through the recorded position of the probe, and
the at least one second point comprises four second points each corresponding to the four vertices of the polygon disposed in the second plane transverse to the current direction of the probe, so that the central area of the polygon is crossed by the straight line parallel to the current direction of the probe and passing through the current position of the probe.

5. The method according to claim 1, wherein:
a first geometric shape is associated with the at least one first point point, the first geometric shape being projected in augmented reality during the projection step, in augmented reality on the at least one displayed image, of the at least one first point, and
a second geometric shape is associated with the at least one second point, the second geometric shape being projected in augmented reality during the projection step, in augmented reality on the at least one displayed image, of the at least one second point.

6. The method according to claim 1, wherein a straight line segment connecting the at least one first point to the at least one second point is displayed as a function of a distance between the at least one first point and the at least one second point.

7. The method according to claim 1, wherein the predetermined point of the surface of the body is predetermined so that a position and an orientation, in the fixed reference frame relative to the patient's body, the surface of the support disposed at the predetermined point is stable over time, and repeatable, i.e. when the step of placing the support at the predetermined point of the surface of the patient's body is repeated several times in succession, a standard deviation of the position of the surface of the support is less than a deviation in position and a standard deviation of the orientation of the surface of the support is less than a deviation in orientation.

8. The method according to claim 1, the recorded position and the recorded direction of the probe being defined by a first user when the probe is held in the hand of the first user in a current position and according to a current direction, the step of determining the current position and the current direction of the probe in the reference frame, from the second markers identifiable on the at least one image comprising a step of recording the current position of the probe and the current orientation.

9. A system for guiding a user holding in one hand a probe configured for ultrasound observation of a body of a patient, the system comprising the probe provided with a first object secured to the probe, the first object comprising first markers on the surface of the first object, the system also comprising a second object comprising second markers on the surface of the second object, the system comprising a support placed at a predetermined point of the surface of the patient's body observed with the probe, the second object being placed on a surface of the support configured to maintain the second object in a predetermined position and orientation of the second object relative to a fixed reference frame relative to the patient's body, the system further comprising a camera configured to observe in its field the probe, the first object, and the second object, the system comprising a display device configured to receive at least one image acquired by the camera, the display device being configured to display the at least one image acquired by the camera, and to display in the at least one image in augmented reality at least one first point corresponding to a vertex of a polygon disposed in a first plane transverse to a recorded direction of the probe, so that a central area of the polygon is crossed by a straight line parallel to the recorded direction of the probe and passing through the recorded position of the probe, and to display in the at least one image in augmented reality at least one second point corresponding to the vertex of the polygon disposed in a second plane transverse to the current direction of the probe, so that a central area of the polygon is crossed by a straight line parallel to the current direction of the probe and passing through the current position of the probe, the user holding the probe in a hand being guided by the display to make the at least one first point coincide with the at least one second point in the at least one displayed image, by displacing the probe to a new current position and a new current direction so as to gradually make the new current position and the new current direction of the probe coincide with the recorded position and the recorded direction of the probe.
